(19) United States
(12) Patent Application Publication
(10) Pub. No.: US 2008/0200308 A1
(43) Pub. Date: Aug. 21, 2008

(54) PORTABLE KIT, DEVICE AND METHOD FOR THE EXECUTION OF CARDIOPULMONARY TESTS UNDER STRAIN, WITH THE USE OF A NORMAL BICYCLE

(75) Inventors: Fabio PEDRINI, Casalecchio di Reno (Bologna) (IT); Paolo BRUGNOLI, Pavona di Albano (Roma) (IT)

Correspondence Address: HESLIN ROTHENBERG FARLEY & MESITI PC, 5 COLUMBIA CIRCLE, ALBANY, NY, 12203, US

(73) Assignee: COSMED ENGINEERING S.r.l, Pavona di Albano (Roma) (IT)

(21) Appl. No.: 12/034,044

(22) Filed: Feb. 20, 2008

(30) Foreign Application Priority Data
Feb. 21, 2007 (IT) TO2007A000123

(51) Int. Cl. A63B 69/16; A61B 5/08

(52) U.S. Cl. 482/6; 600/529

(57) ABSTRACT

A device for the execution of cardiopulmonary tests under strain includes a device for measuring the respiratory activity of a user and physical exercise equipment to be used in combination with the measuring device. The physical exercise equipment is a portable training tool, to be used in combination with a normal bicycle in stationary position.

### CROSS-REFERENCE TO RELATED APPLICATIONS

This application claims priority from Italian patent application No. TO2007A000123, filed on Feb. 21, 2007, the entire disclosure of which is incorporated herein by reference.

### BACKGROUND OF THE INVENTION

The present invention relates to devices for executing cardiopulmonary tests under strain.

The applicant COSMED Engineering S.r.l. constitutes the design and development centre of COSMED S.r.l., which has produced and sold devices adapted to measure the oxygen consumption of a user for some time (see for example US 2006/0005836 A1). Devices of this type are also used for executing tests under strain, with the combined use of physical exercise equipment, typically of the “cycle ergometer” type, that are relatively cumbersome, not transportable and expensive.

### SUMMARY OF THE INVENTION

It is an object of the present invention to allow the possibility to carry out cardiopulmonary tests under strain with extremely simple means, of reduced dimensions and reduced pure cost, in order to make it possible for the individual user to undergo such tests, with extreme ease and convenience, without the need to provide sophisticated equipment in special places.

In order to achieve this aim, the object of this invention is a portable kit comprising:
a training tool, folding or dismountable in a configuration of reduced dimensions, adapted to allow the use of a normal bicycle in a stationary position by a user, said tool including:
support means for the bicycle in use position, leaving the rear wheel of the bicycle the possibility to turn, and
electronically controlled actuator means, for applying a variable braking force to the rear wheel of the bicycle,

said portable kit also comprising:
a portable device for measuring the cardiopulmonary activity of the user, including measuring means connected to a mask or breathing mouthpiece that can be worn by the user, and
an electronic control unit operatively connected both to said actuator means and said measuring means.

Thanks to the above-mentioned characteristics, the invention allows the user to have an extremely simple and transportable structure with ease in any place, that allows the execution of cardiopulmonary tests under strain with the use of a normal bicycle.

In the case of sport cyclists, professionals or amateurs, the invention hence allows the cyclist the advantage of being able to carry out the necessary tests under strain not with a machine, but with the same bicycle then to be used for competitions.

In any case, thanks to the invention, a user can undergo the test with ease and convenience, once a normal bicycle is available, using the portable training tool and the portable measuring device. The portable training tool can be created, in a known manner, as a conventional tool for the use of a normal bicycle in a stationary position. Tools of this type have been sold and used for some time. In a typical embodiment, the portable training tool comprises a frame, folding or dismountable in a configuration of reduced dimensions, for ease of transport. Such frame is provided to support the bicycle in use position, leaving the rear wheel the possibility to turn. The tool also comprises a controllable braking device, borne by said frame, and adapted to make friction contact with the tyre of said bicycle wheel for applying a braking force to it.

According to the invention, the actuator means with which the portable training tool is provided for regulating the braking force on the bicycle wheel are operatively connected to the electronic control unit which is also operatively connected to the measuring means of the user's cardiopulmonary activity. The electronic control unit is arranged to send a command signal to said actuator means for controlling the application of a required value of the braking force and is also arranged to receive a feedback signal from said actuator means, indicative of the braking force applied by it. Based on the data set by the user, the electronic control unit can therefore control said actuator means of applying the braking force according to a closed ring logic.

The electronic control unit is associated with a display and/or printing unit of the measuring results of the cardiopulmonary parameters, including the oxygen consumption.

In a preferred embodiment, the above-mentioned electronic control unit, with said display and/or printing unit, and the above-mentioned interface unit are both associated with the measuring device. In such an embodiment, the device comprises a case carrying a keyboard and a display, as well as a printing unit for printing a report concerning the test results.

A fundamental characteristic of the invention lies in its portability. Such a characteristic is obtained thanks to the fact that both the measuring device and the training tool, thanks to its foldable and/or dismountable nature, are portable. The key idea for obtaining such an advantage lies in the fact that the physical exercise equipment used in combination with the measuring device of the respiratory activity is not constituted of a complete dedicated machine, but a portable tool, to be used in combination with a normal bicycle.

Thanks to the above-mentioned characteristics, the user can take the kit with them according to the invention, constituted of the training tool and the measuring device, with convenience and ease, in order to be able to carry out the cardiopulmonary test under strain, at any time and anywhere, once there is any normal bicycle available.

Furthermore, in the case of a sports cyclist, professional or amateur, it will hence be possible to carry out the cardiopulmonary test on one's own bicycle, so as to make the test more indicative compared to that which can be carried out with a cycle ergometer type machine.

According to a further preferred characteristic, the kit also comprises a measuring device of the cardiac activity, electrically connectable or with wireless communication with the above-mentioned electronic control unit.

### BRIEF DESCRIPTION OF THE DRAWINGS

Further characteristics and advantages of the invention will be clear from the following description with reference to the attached drawings, provided by way of a non-limitative example, in which:

FIG. 1 is a schematic perspective view showing the use of a kit according to the invention,

FIG. 2 is a enlarged perspective view of the training tool,

FIG. 2A is a block diagram of the device according to the invention, and

FIG. 3 is an enlarged perspective view of the measuring device of the cardiopulmonary activity, which is part of the device according to the invention.

### DETAILED DESCRIPTION OF THE INVENTION

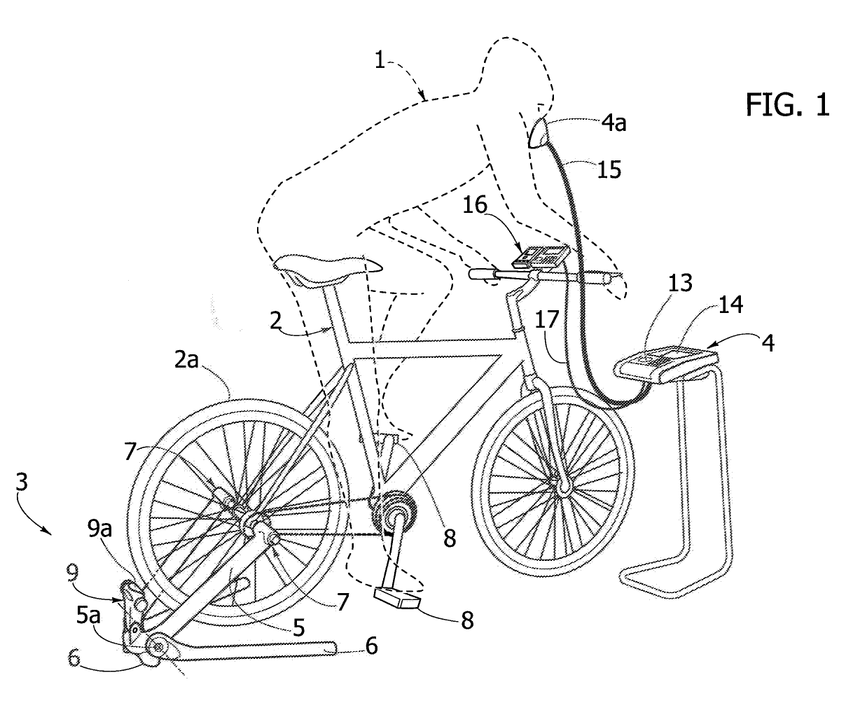

With reference to FIG. 1, a user 1 undergoes a respiration test under strain making use of a normal bicycle 2, with the aid of the kit according to the invention, including a portable training tool 3 and a portable measuring device 4 of the cardiopulmonary activity. The portable tool 3 is of any known type that allows the use of a normal bicycle in stationary position. In the case of the example depicted, the tool comprises a frame 5 provided with feet 6 that rest on the ground and supports 7 for supporting the hub of the rear wheel of the bicycle 2 freely pivotable, giving the bicycle stability at the same time. In such a condition, the user 1 can use the bicycle 2 in stationary position imparting rotation to the rear wheel 2a of the bicycle 2 using the pedals 8. The frame 5 comprises joints 5a that allow it to be folded and/or dismounted into a condition of reduced dimensions, for ease of transport.

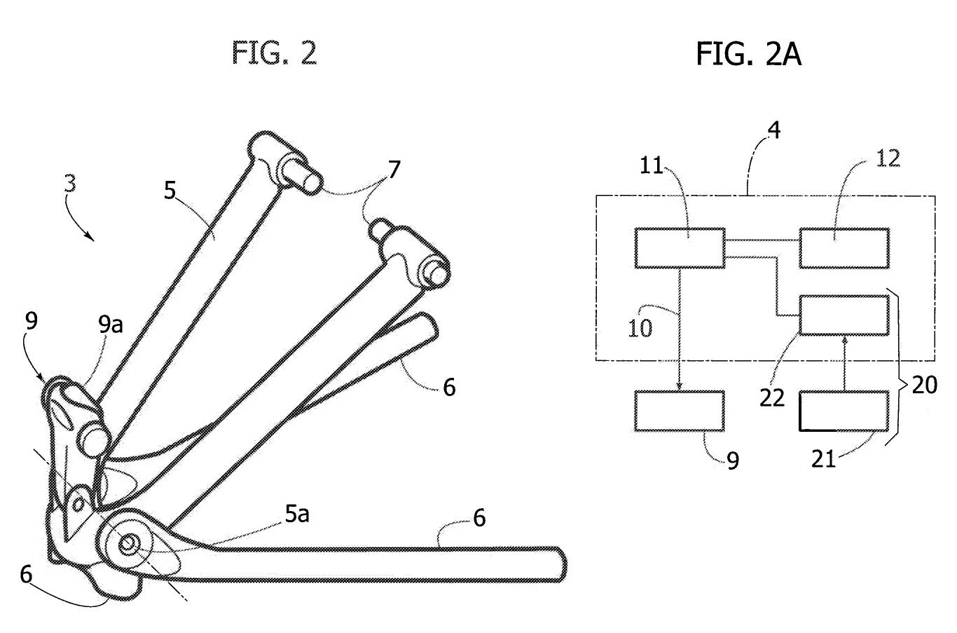

The device 3 also comprises electrically controlled actuator means 9 of any known type, that is activated by pressing a roller 9a freely pivotable against the tread of the tyre of the rear wheel 2a, in order to exert a braking force on the wheel. Such a braking force can be modified by varying the electrical signal 10 that is sent to the actuator means 9 by an electronic control unit 11 (FIG. 2A). The electronic control unit is operatively connected for example using a wire (or alternatively through a wireless connection) to the measuring means 12, being part of the measuring device 4.

In the case of the example depicted in FIGS. 1-3, the electronic control unit 11 is contained in the case of the measuring device 4, which is also provided with an interface unit with the user, including a keyboard 13, a display unit 13, and potentially a printing unit (not shown in the drawings) which is contained in the case of the device 4 and is adapted to issue a sheet or printed tape of the test results through an opening exit. The measuring means 12 of the device 4 is connected using a flexible pipe 15 with a mask or breathing mouthpiece 4a and allowing the device 4 to show the characteristics of the user's respiratory activity.

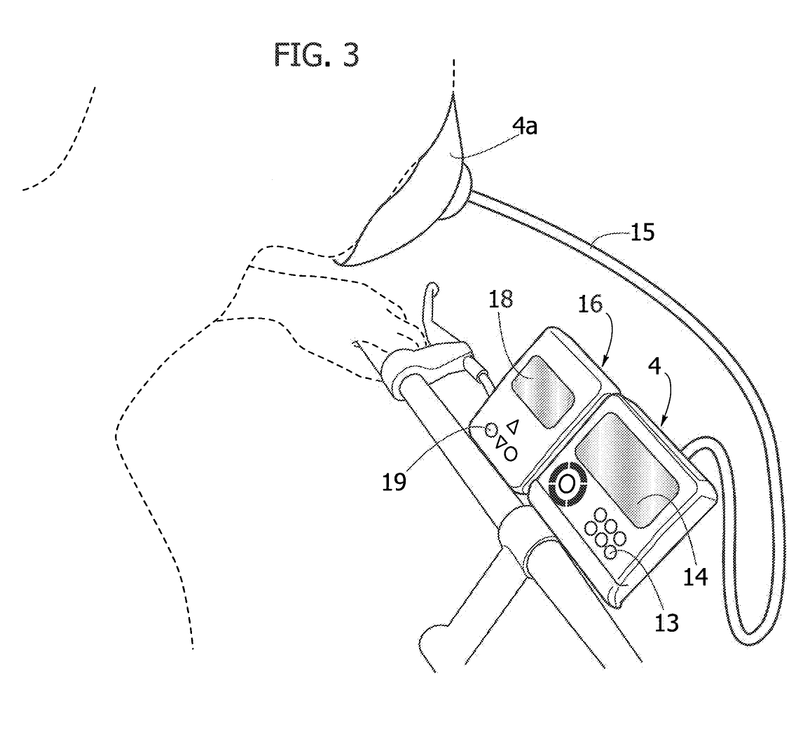

In the case of the example shown in FIG. 1, as well as the main electronic control unit 11 shown in FIG. 2A and contained inside the case of the measuring device 4, a second electronic control module 16 is provided which is connected, for example via a cable 17, with a first control module 11 and that also bears (see FIG. 3) a display (18) and a keyboard (19), for setting the braking force on the wheel 2a.

FIG. 1 refers to the case in which the second module 16 is provided with means for its assembly on the bicycle handlebar, whilst the measuring device 4 is provided to be placed on any support surface adjacent to the test position.

FIG. 3 shows an alternative configuration in which both the modules 11 and 16 are assembled on the bicycle handlebar.

As a further alternative, the second unit 16 can be eliminated and the control unit 11 can be in direct communication with the actuator means 9.

Furthermore, the kit for executing the test also comprises measuring means 20 of the user's cardiac activity, also connectable to the electronic control unit 11 of the measuring device, and including, in a known manner, sensor means 21 associable with the user's chest and receiver means 22 for receiving the signals coming from the sensor means 21, for example via wireless communication.

The embodiment details of the measuring device 4 and the relative control unit are not shown here, as they can be carried out in any known manner, and given that they, per se, are not part of the invention. The elimination of these details from the drawings also makes the latter clearer and easier to understand. As indicated, an example of a device of this type is provided by the document US 2006/0005836.

In use, the user can set, for example using the keyboard 19 of the second control module 16, or directly using the keyboard 13 of the device 4 in case the unit 16 is not provided, a desired value or a desired programmed variation in the braking force to be applied to the bicycle wheel, after which the test can begin, with the measuring device 4 that carries out the detection of the user's cardiopulmonary activity during the exercise.

As already indicated above, the fundamental characteristic of the invention is that of allowing the user to carry out a cardiopulmonary test under strain with ease and convenience anywhere, with the aid of portable equipment. This result is obtained as the measuring unit 4 is of a portable type and is used not in combination with a whole machine dedicated to that aim, but using the simple portable equipment 3 that can be used in combination with any normal bicycle.

Naturally, various modifications to the construction details and the embodiments can be possible without departing from the principle of the invention, referring to that which is described and shown by way of example.

What is claimed is:

1. A portable kit for carrying out cardiopulmonary tests, comprising:
a training tool, folding or dismountable in a configuration of reduced dimensions easily transportable, adapted to allow the use of a normal bicycle in an stationary position by a user, said tool including:
support means to support the bicycle in an in-use position, leaving the rear wheel of the bicycle the possibility to turn, and
electronically controlled actuator means, for applying a variable braking force to the rear wheel of the bicycle,
a portable device for measuring the cardiopulmonary activity of the user, including measuring means connected to a mask or breathing mouthpiece wearable by the user, and an electronic control unit operatively connected both to said actuator means, and said measuring means.

2. The portable kit according to claim 1, wherein said electronic control unit is arranged to send to said actuator means a control signal to control the application of a desired value of braking force, and is also arranged to receive from said actuator means a feedback signal, indicating the braking force applied by said actuating means.

3. The portable kit according to claim 1, wherein said electronic control unit is operatively connected to an interface unit with the user, through which the user can set a pre-determined value of the braking force applied by said actuator means, or a pre-determined programmed variation in the braking force.

4. The portable kit according to claim 1, wherein said electronic control unit is combined with at least one of a display unit and a printing unit for indicating results of a measurement by said measuring means.

5. The portable kit according to claim 3, wherein said electronic control unit, with said at least one of a display unit and a printing unit, and said interface unit are associated with said measuring device.

6. The portable kit according to claim 5, wherein said electronic control unit is integrated in a case of said measuring device.

7. The portable kit according to claim 5, wherein said interface unit includes a keyboard.

8. The portable kit according to claim 5, wherein said at least one of a display unit and a printing unit includes a display associated with a case of said measuring device.

9. The portable kit according to claim 1, wherein said control unit comprises a first control module associated with said measuring device and a second control module physically separated from said measuring device and operatively connected to said first module, said second module having a respective keyboard and a respective display for setting and displaying a desired value of the braking force or a desired variation program of the braking force.

10. The portable kit according to claim 9, wherein at least one of said first module and said second module is provided with means for assembling said at least one of said first module and said second module on the bicycle handlebar.

11. The portable kit according to claim 1, wherein said portable training tool comprises a frame for supporting the rear wheel freely pivoting of the bicycle, and an electrically controlled braking device, borne by said frame and adapted to enter into contact with the bicycle tire in order to apply a braking force on it.

12. The portable kit according to claim 1, wherein said portable device for measuring the user's cardiopulmonary activity includes measuring means of the user's cardiac activity, comprising sensor means applicable to the user's chest and receiver means of the signals coming from said sensor means.

13. A device for executing a cardiopulmonary test under strain, comprising a device for measuring the user's respiratory activity, including measuring means connected to a mask or a breathing mouthpiece wearable by the user, and physical exercise equipment to be used in combination with the measuring device,
wherein said physical exercise equipment is a portable training tool, to be used in combination with a normal bicycle in stationary position, and wherein said measuring device is portable.

14. A method for executing respiratory tests under strain, comprising a measurement stage of the characteristics of the cardiopulmonary activity of a user through a measuring device connected to a breathing machine that is worn by the user, in which such measurement operation is carried out during the execution of physical exercise by the user with the aid of physical exercise equipment,
wherein said physical exercise equipment is a foldable or dismountable training tool in a configuration of reduced dimensions easily transportable, adapted to allow the use of a normal bicycle in stationary position by a user.
